(19) United States
(12) Patent Application Publication
(10) Pub. No.: US 2005/0228473 A1
(43) Pub. Date: Oct. 13, 2005

(54) Device and method for delivering a treatment to an artery

(76) Inventor: David Brown, Bronxville, NY (US)

Correspondence Address: The Law Office of Steven G. Roeder, 5560 Chelsea Avenue, La Jolla, CA, 92037, US

(21) Appl. No.: 10/818,893

(22) Filed: Apr. 5, 2004

(51) Int. Cl. A61F 2/06

(52) U.S. Cl. 623/1.2; 623/1.42

(57) ABSTRACT

A treatment device (10) for treating a treatment site (13) within a vessel (12) includes a frame (16) that is positioned in the vessel (12) at or near the treatment site (13). The vessel (12), for example, can be an artery or other internal passageway within a patient (20). The vessel (12) has a preplacement site inner diameter (28) near the treatment site (13). The treatment device (10) includes a frame (16) that moves from a contracted first configuration (30) to an expanded second configuration (236). The frame (16) has a frame outer diameter (240) at the second configuration (236) that is approximately equal to the preplacement site inner diameter (28). The frame (16) can deliver a treatment (242) to the treatment site (13).

### BACKGROUND

The process of atherosclerosis causes fatty deposits (plaque) to accumulate in the walls of arteries. As the process becomes more advanced, the fatty deposits begin to encroach on the lumen of the artery resulting in blockages (stenosis) of varying degrees. Blockages of approximately 70% or more of the normal vessel diameter are associated with inadequate blood flow to the heart muscle during stress or exercise resulting in the symptom known as angina pectoris (chest pain). The current treatment of such blockages usually involves placement of a metallic support device called a stent at the site of the blockage to enlarge the lumen and restore blood flow to normal, thereby eliminating the symptoms of angina.

The stent is delivered to the site of stenosis mounted on a balloon catheter that is unexpanded. When the stent is in the proper location, the balloon catheter is inflated to high pressure (12-20 atmospheres), stretching the artery open and imbedding the stent in the vessel wall. The inherent radial strength of the stent maintains the diameter achieved during balloon inflation after the balloon catheter is deflated and removed from the body.

The arteries also contain many blockages of less severity that do not impair blood flow to the heart muscle during stress or exercise. However, when these plaques become inflamed, they become susceptible to rupturing open. Plaque rupture initiates blood clotting (thrombosis) at the site of rupture. A sufficiently large clot can occlude the vessel and eliminate blood flow through the vessel. This can result in a heart attack or sudden death.

Newer technologies are being developed to detect these inflamed, non-flow limiting stenoses that are vulnerable to rupture including, but not limited to, thermography, palpography, near infrared and Raman spectrospcopy, optical coherence tomography and magnetic resonance imaging. However, once a vulnerable plaque is identified, there is no specific treatment available. Because the vulnerable plaque is usually not associated with a stenosis severe enough to impair blood flow, the structural support provided by a stent is unnecessary and potentially deleterious. The high pressure deployment of a stent is associated with significant arterial trauma that can result in the perforation or dissection of an artery. Furthermore, the truama incited by stent placement can induce a scarring response that can cause the artery to become severely narrowed or even occluded. Thus, stenting of mildly narrowed vulnerable plaque may convert an asymptomatic patient into one with an acute complication requiring emergency cardiac surgery or into one with a more severe blockage from scarring resulting in angina and the need for additional procedures and/or medications. Additionally, stent placement causes inflammation acutely and thus could exacerbate or amplify the underlying inflammation in the vulnerable plaque.

### SUMMARY

The present invention is directed to a treatment device for treating a treatment site of an internal vessel of a mammal, such as a human being. The vessel, for example, can be an artery or other internal passageway within a patient. The vessel has a preplacement site inner diameter at a placement site prior to placement of the treatment device. The treatment device includes a frame that expands from a contracted, first configuration to an expanded, second configuration.

In one embodiment, the frame has a frame outer diameter at the second configuration that is approximately equal to the preplacement site inner diameter. In alternative non-exclusive embodiments, the frame outer diameter at the second configuration is greater than or equal to approximately 0.1, 0.2, 0.3, 0.5, 0.75, 1, 1.5, 2, 3, 4, 5, 6, 7, 8, 9, 10, 12, 14, 16, 18, or 20 percent of the preplacement site inner diameter.

In one embodiment, the treatment device delivers a treatment to the treatment site. For example, the treatment device can include a coating that emits a treatment to the treatment site. In an alternative, non-exclusive embodiment, the treatment can include a drug or molecule that is emitted from the device into the bloodstream to be targeted to a treatment site downstream from the device.

In one embodiment, the frame includes a series of interconnected tubular shaped coils. In the expanded second configuration, the frarhe closely replicates the original lumen of the vessel at the placement site, and exerts only enough outward radial force to keep it in position at the placement site in the vessel.

The frame can be made of a resilient material and can be formed so that absent some restraint, each band expands from the first configuration to the second configuration. The amount that each band expands, absent restraint, can be controlled during the manufacture of the frame to suit the particular use of the treatment device.

In certain embodiments, the treatment device can be used to treat vulnerable or inflamed plaque with minimal trauma. For example, rather than increasing the arterial lumen like a stent, in certain embodiments, the treatment device will conform to the lumen and will exert only the minimal radial force required to prevent the treatment device from migrating down the artery.

The present invention is also directed to a method for treating a treatment site in a vessel.

### BRIEF DESCRIPTION OF THE DRAWINGS

FIG. 1 is a side illustration of a portion of a vessel having a placement site, a device positioner, and a treatment device in a first configuration positioned away from the placement site;

FIG. 2A is a side view of the treatment device of FIG. 1 in the first configuration;

FIG. 2B is a side view of the treatment device of FIG. 1 in a second configuration;

FIG. 2C is a side view of another embodiment of a treatment device having features of the present invention;

FIG. 3A is a side illustration of the vessel, the device positioner, and the treatment device in the first configuration positioned near the placement site;

FIG. 3B is a side illustration of the vessel, the device positioner, and the treatment device in the second configuration positioned at the placement site;

FIG. 3C is a side illustration of the vessel and the treatment device in the second configuration at the placement site; and

FIG. 4 is a side illustration of another vessel with the treatment device at the placement site.

### DESCRIPTION

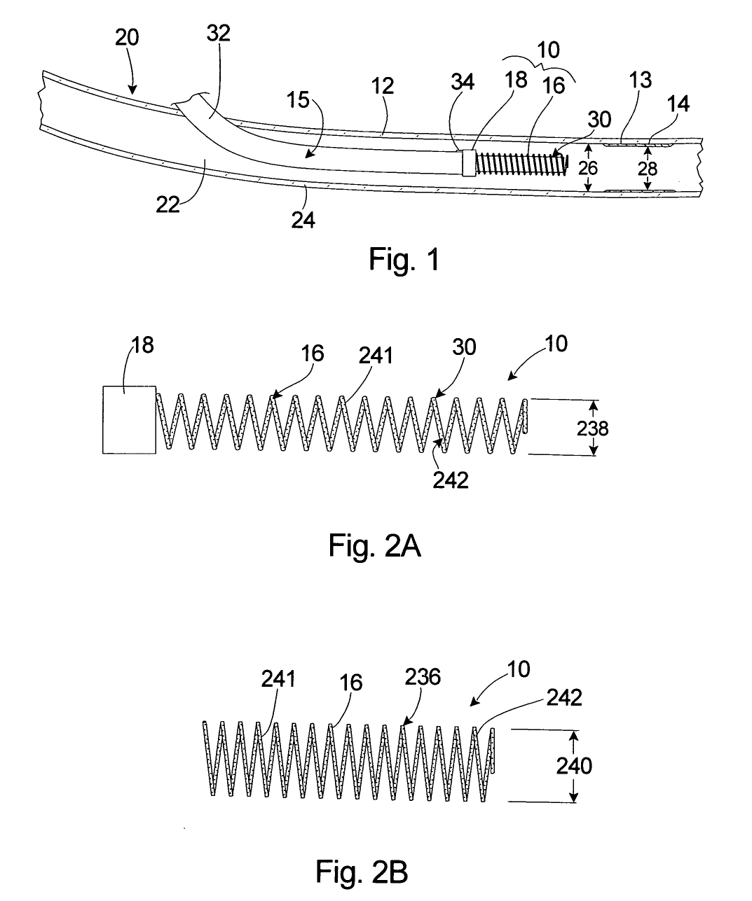

FIG. 1 is a side plan illustration of a first embodiment of a treatment device 10 having features of the present invention, a vessel 12 having a treatment site 13, a placement site 14, and a device positioner 15. In one embodiment, the treatment device 10 is used to treat the treatment site 13. The design of the treatment device 10 can vary according to the requirements of the treatment site 13. In the embodiment illustrated in FIG. 1, the treatment device 10 includes a frame 16 and a retainer 18.

The type of vessel 12 and treatment site 13 can vary. For example, the vessel 12 can be an artery of a mammal 20 (only a portion illustrated in FIG. 1), such as a human being. Alternatively, for example, the vessel 12 can be another body passageway in the vascular system or an organ. In one embodiment, the vessel 12 includes a lumen 22 and a vessel wall 24. In one embodiment, the treatment site 13 is a slight fatty deposit of material, e.g. plaque, on the inner lining of the vessel wall 24. Alternatively, for example, the treatment site 13 can be an internal organ (brain or liver) or pathological process (tumor or malignancy) that is positioned downstream in the vessel 12 from the placement site 14.

In the embodiment illustrated in FIG. 1, the treatment site 13 is the same as the placement site 14, and the lumen 22 of the vessel 12 includes a lumen inner diameter 26 near the placement site 14 and a preplacement site inner diameter 28 at the placement site 14 prior to placement of the treatment device 10 and/or any other treatment of the treatment site 13. In alternative, non-exclusive embodiments, the present invention is useful when the preplacement site inner diameter 28 is within approximately 0.5, 1, 2, 3, 4, 5, 7, 10, 12, 14, 16, 18, or 20, 30, 40, 50 or 60 percent of the lumen inner diameter 26. Stated another way, in alternative, non-exclusive embodiments, the present invention is useful when the preplacement site inner diameter 28 stenosis is ≦60% of the lumen inner diameter 26.

In one embodiment, the treatment device 10 is used to treat what would typically be considered an angiographically normal or minimally abnormal vessel wall 24.

FIG. 1 illustrates that the frame 16 of the treatment device 10 is initially in a retracted first configuration 30 on the device positioner 15. In this embodiment, the device positioner 15 is maneuvered by a doctor to move the treatment device 10 in the vessel 12 and position the treatment device 10 at or adjacent to the treatment site 13 and the placement site 14. The design of the device positioner 15 can vary according to the design of the treatment device 10. In FIG. 1, the device positioner 15 is a catheter 32 that includes a retractor 34. In this embodiment, the retractor 34 includes a hook that is selectively moved by the doctor to selectively remove the retainer 18. The catheter 32 can be positioned over a guidewire (not shown) in the vessel 12. Further, the catheter 32 can be introduced into the vessel 12 wherever it is most convenient to do so.

It is to be understood that other means and methods for placing the frame 16 at the treatment site 13 may occur to one skilled in the art and the invention is not restricted to the catheter introduction and placement system described herein.

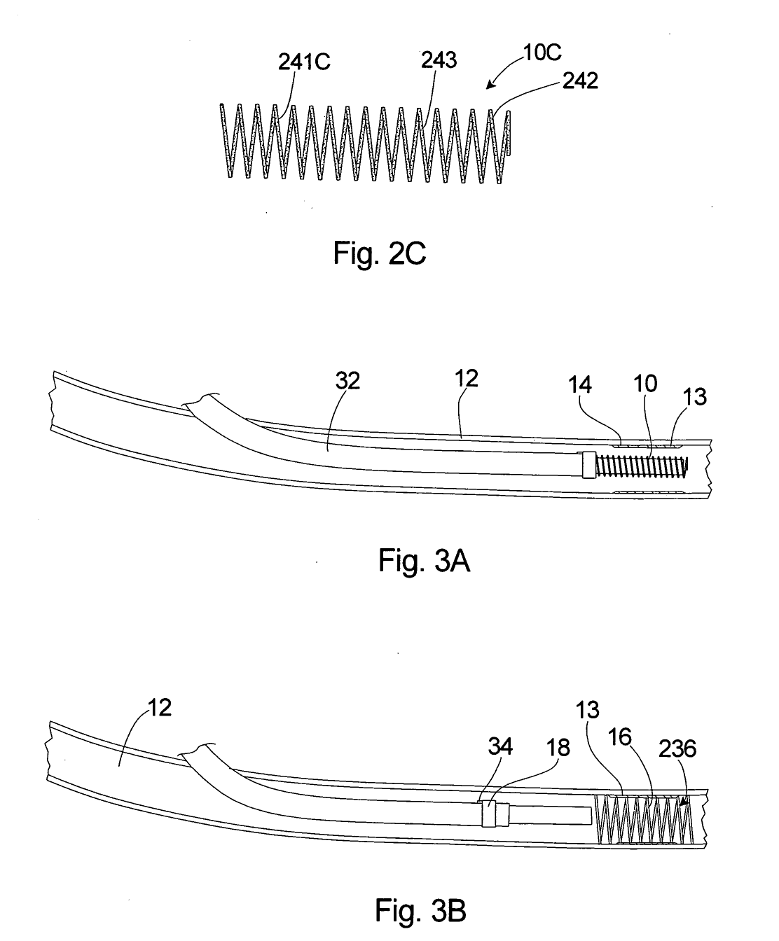

FIG. 2A is a side view of the treatment device 10 including the retainer 18 and the frame 16 in the first configuration 30. The frame 16 may be fabricated in a large range of diameters and overall lengths. In the first configuration 30, the frame 16 has a first frame configuration outer diameter 238 which is less than the lumen inner diameter 26 and the preplacement site inner diameter 28 (illustrated in FIG. 1). With this design, in the first configuration 30, the treatment device 10 can be moved atraumatically in the vessel 12 to the treatment site 14 (illustrated in FIG. 1). In one embodiment, in the first configuration 30, the treatment device 10 can be straightened to be substantially linear.

FIG. 2B is a side view of the treatment device 10 without the retainer and with the frame 16 in an expanded second configuration 236, e.g. the configuration the treatment device 10 will have in the vessel 10 after the treatment device 10 is deployed in the vessel 10. In one embodiment, in the second configuration 236, the frame 16 is at maximum expansion.

In one embodiment, in the second configuration 236, the frame 16 has a second frame outer diameter 240 which is approximately equal to the preplacement site inner diameter 28 of the vessel 12 at the placement site 14 (illustrated in FIG. 1). With this design, the frame 16 can be placed and retained at the placement site 14 without significantly modifying or altering the diameter at the placement site 14. In alternative, non-exclusive embodiments, the second frame outer diameter 240 is no greater than approximately 1, 5, 10, 15 or 20 percent of the preplacement site inner diameter 28.

Further, in non-exclusive examples, the frame 16 in the second configuration may range in length from about ten millimeters to forty millimeters (10.0 mm-40.0 mm) and have a second frame outer diameter 240 of between approximately 2 and 40 millimeters. However, the frame 16 can have other lengths and/or diameters.

Referring to both FIGS. 2A and 2B, in this embodiment, the frame 16 is helical shaped and includes a plurality of continuous coils 241. In this embodiment, during movement of the frame 16 from the first configuration 30 to the second configuration 236, the diameter of the coils 241 increases and the length of the frame 16 decreases. While a helical coil construction is illustrated in FIGS. 2A and 2B, it should be understood that other constructions may also be employed without departing from the spirit and scope of the invention. For example, in an alternative embodiment, the frame 16 can include a series of separate tubular shaped bands that are interconnected by one or more elongated strips. Additionally, the frame 16 can be hollow.

In one embodiment, the frame 16 is made of a shape memory material that remains in the first configuration 30 when retained by the retainer 18 and that moves to the second configuration 236 when the retainer 18 is removed. In this embodiment, for example, the frame 16 can be made of Nitinol.

In one embodiment, in order to form the frame 16 so that it will assume the second configuration 236 when the retainer 18 is removed, a length of shape memory material wire is coiled around a mandrel (not shown) so that it has the second configuration 236. The wire is then heated until its crystal structure assumes the second configuration 236. The frame 16 can now be bent and extended into the relatively smaller diameter first configuration 30. Because the frame 16 is formed of the shape memory material, it will revert to the second configuration 236 when the retainer 18 is removed.

In the embodiment illustrated in FIG. 2A, the retainer 18 retains the frame 16 in the first configuration 30 during positioning of the treatment device 10 in the vessel 12. In one embodiment, the retainer 18 is a tubular shaped sheath that encircles all or a portion of the frame 16. In FIG. 2A, the sheath encircles only a portion of the frame 16. In this embodiment, the device positioner 15 (illustrated in FIG. 1) is used to remove the retainer 18 when the treatment device 10 is within the placement site 14.

Alternatively, for example, the retainer 18 can be an adhesive that secures the frame 16 to the device positioner 15 and retains the frame 16 in the first configuration 30. Still alternatively, the treatment device 10 can be designed without the retainer 18.

In one embodiment, the frame 16 is designed so that the pitch of the coils can be controlled by controlling the speed at which the retainer 18 is removed from the frame 16. With this design, by varying the removal rate of the retainer 18 with the retractor 34, the distance between the coils 241 can be modified. Slow withdrawal would allow the coils 241 to be distantly spaced resulting in a lighter concentration of a treatment 242 (illustrated as shading). More rapid withdrawal during unsheathing would result more closely spaced coils 241 covering a shorter length of vessel 12 and greater concentrations of the treatment 242.

In another embodiment, for example, the frame 16 is made from a shape memory alloy, e.g. Nitinol, with a transition temperature. In this embodiment, the frame 16, when heated to the transition temperature, expands to the second configuration 236. In one embodiment, the transition temperature is in the range of between approximately 115-125 degrees Fahrenheit. However, for Nitinol, the transition temperature can be manipulated over a wide range by altering the nickel-titanium ratio, by adding small amounts of other elements, and by varying deformation and annealing processes.

In this embodiment, in order to form the frame 16 so that it will assume the second configuration 236 when heated to its transition temperature, a length of shape memory wire is coiled around a mandrel at room temperature so that it has the larger diameter second configuration 236. Subsequently, the frame 16 is then heated until its crystal structure assumes its high-temperature austenite configuration. Next, the frame 16 is cooled so that the atoms in the metal rearrange themselves into a crystal structure known as martensite. The frame 16 may now be bent and extended into the relatively smaller diameter first configuration 30. Because the frame 16 is formed of a shape memory material, it will revert to the large diameter second configuration 236 when the frame 16 is later heated to a temperature at which the crystal structure reverts to the parent phase. The frame 16 will retain this shape unless it is cooled below a temperature at which martensite transformation of the shape memory material occurs. Because this transformation only begins at temperatures well below normal body temperature, the frame 16 should not return to the first configuration 30 when positioned in the body.

In this embodiment, to expand the frame 16, it is necessary to heat the frame 16 to a temperature at which the shape memory material reaches its transition temperature and austenite transformation occurs. In one embodiment, this can be accomplished by injecting a hot fluid from the catheter 32 onto the frame 16 to trigger expansion of frame 16. As the frame 16 reaches its transition temperature (125-130 degrees Fahrenheit) it promptly reassumes the second configuration 236. Once the frame 16 is expanded to the second configuration 236, the frame 16 is pressed against the vessel wall 24 and the catheter 32 can be removed. Alternatively, for example, expansion may also be produced by induction or microwave warming of the frame 16 through the skin, or by a separate heating device temporarily positioned in or near the vessel 12.

In yet another embodiment, the frame 16 is formed from a material that is moved by a balloon catheter or other device from the first configuration 30 to the second configuration 236.

In one embodiment, the treatment device 10 delivers the treatment 242, e.g. local drug delivery, to the treatment site 14. Stated another way, the frame 16 can emit and/or deliver a treatment 242 to the treatment site 13. For example, the frame 16 can be coated with one or more treatments 242, e.g. drugs or therapeutic agents or molecules that have beneficial effects on the treatment site 13. The treatments 242 may be bound to the frame 16 directly or with a polymer. Alternatively, the polymer coating the frame can consist of therapeutic molecules that are released at the treatment site 13 as the polymer degrades. Alternatively, the material of the frame 16 can emit the treatment 242.

Alternatively, the frame can be entirely biodegradable, dissolving over a period of time after or coincident with the delivery of a treatment. Alternative, non-exclusive examples of possible biodegradable materials include poly(L-lactic acid) and poly(D,L-lactic-co-glycolic acid) (PLGA).

The design of the treatment 242 can depend upon the treatment site 13. For example, the treatment 242 can prevent plaque rupture, stabilize vulnerable plaque, cause a reduction in plaque volume, or inhibit new plaque development. In one embodiment, the treatment 242 can include agents that stimulate reverse cholesterol transport, e.g. HDL (high density lipoprotein), ApoA1 (apolipoprotein A1), or ApoA2 (apolipoprotein A2) (natural occurring and/or synthetic versions). Alternatively, for example, the treatment 242 can cause an increase in collagen synthesis. For example, the treatment 242 can include steroidal anti-inflammatory compounds (dexamethasone), non-steroidal anti-inflammatory compounds (salicylic acid), matrix metalloproteinase inhibitors (tetracycline), antibiotics (tetracycline), or a protease inhibitor. Further, the treatment 242 can be a compound that increases the number of smooth cells, or reduces the number of macrophages. Further, the treatment can include inhibitors of nuclear factor kappa B, inhibitors of p38 mitogen activator protein kinase or agonists of peroxisome proliferator-activated receptor-gamma. Each compound can include a drug, therapy or molecule. Each treatment may contain a single therapy or a combination of therapies.

FIG. 2C illustrates yet another embodiment of the treatment device 10C. In this embodiment, the coils 241C of the treatment device 10C are hollow e.g. a hollow tube, and include a plurality of device apertures 243 that extend into the hollow coils 241C. With this design, the hollow coils 241C can be filled with a treatment 242 that is released via the device apertures 243. In this design, the coils 241C can also be coated with the treatment 242.

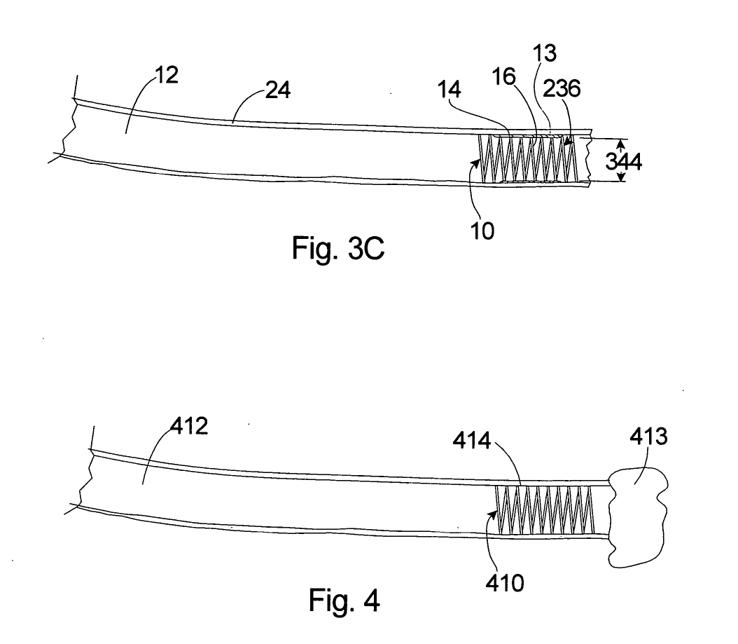

FIG. 3A illustrates that the catheter 32 has been moved in the vessel 12 so that the treatment device 10 is positioned within the lumen at the placement site 14.

FIG. 3B illustrates that the retractor 34 has removed the retainer 18 and the frame 16 has expanded to the second configuration 236 in the vessel 12.

FIG. 3C illustrates the vessel 12 without the catheter, and the frame 16 has expanded to the second configuration 236 in the vessel 12.

In one embodiment, after deployment, the treatment device 10 does not provide structural support to the vessel wall 24 and/or does not significantly alter or modify the preplacement site inner diameter 28. Rather, the treatment device when deployed conforms to the internal diameters of the placement site 14 and adjacent artery. With this design, the treatment device 10 does not influence, improve or modify flow in the vessel 12. Further, the treatment device 10 is unlikely to cause dissections or perforations. Moreover, the treatment device 10 does not induce significant trauma to the vessel wall 24 that might result in induction of a significant narrowing as a response to deep vessel injury. In alternative, non-exclusive embodiments, a post placement site inner diameter 344 at the placement site 14 after insertion of the treatment device 10 is approximately 0.5, 1, 2, 3, 4, 5, 7, 10, 12, 14, 16, 18, or 20 percent greater than the preplacement site inner diameter 28 before insertion of the treatment device 10.

FIG. 4 illustrates another situation in which the treatment device 410 is positioned in the vessel 412 at the placement site 414 which is upstream of the treatment site 413. With this design, treatments (not shown in FIG. 4) from the treatment device 410 flow downstream to treat the treatment site 413. In this embodiment, the treatment site 413, for example, can be an internal organ or a pathologic process.

Further, while the particular treatment device 10 as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A treatment device for treating a treatment site of a mammal, the mammal having a vessel that includes a placement site having preplacement site inner diameter prior to treatment, the treatment device comprising:
a frame that moves from a contracted first configuration to an expanded second configuration, the frame having a frame outer diameter at the second configuration that is approximately equal to the preplacement site inner diameter.

2. The treatment device of claim 1 wherein the frame outer diameter at the second configuration is within approximately one percent of the preplacement site inner diameter.

3. The treatment device of claim 1 wherein the frame outer diameter at the second configuration is within approximately five percent of the preplacement site inner diameter.

4. The treatment device of claim 1 wherein the frame outer diameter at the second configuration is within approximately ten percent greater than the preplacement site inner diameter.

5. The treatment device of claim 1 wherein the frame outer diameter at the second configuration is within approximately twenty percent greater than the preplacement site inner diameter.

6. The treatment device of claim 1 wherein the frame includes a coating that emits a treatment to the treatment site.

7. The treatment device of claim 1 wherein the frame is biodegradable.

8. The treatment device of claim 1 wherein the frame is hollow.

9. The treatment device of claim 8 wherein the frame includes a plurality of device apertures that extend into the hollow frame.

10. The treatment device of claim 1 wherein the frame delivers a treatment to the treatment site.

11. The treatment device of claim 10 wherein the treatment is an anti-inflammatory compound.

12. The treatment device of claim 10 wherein the treatment is a non-steroidal anti-inflammatory compound.

13. The treatment device of claim 10 wherein the treatment is a steroidal anti-inflammatory compound.

14. The treatment device of claim 10 wherein the treatment is a protease inhibitor.

15. The treatment device of claim 10 wherein the treatment is a matrix metalloproteinase inhibitor.

16. The treatment device of claim 10 wherein the treatment is an agent that increases collagen synthesis.

17. The treatment device of claim 10 wherein the treatment is an agent that increases the number of smooth muscle cells.

18. The treatment device of claim 10 wherein the treatment is an agent that reduces the number of macrophages.

19. The treatment device of claim 10 wherein the treatment includes at least one of ApoA1 (natural occurring or synthetic versions) or ApoA2 (natural occurring or synthetic versions).

20. The treatment device of claim 10 wherein the treatment is an inhibitor of nuclear factor (NF) kappa B.

21. The treatment device of claim 10 wherein the treatment is an inhibitor of p38 mitogen activator protein (MAP) kinase.

22. The treatment device of claim 10 wherein the treatment is an agonist of peroxisome proliferator-activated receptor (PPAR)-gamma.

23. The treatment device of claim 10 wherein the treatment is an inhibitor of 3-hydroxy-3-methlyglutaryl (HMG) coenzyme A (CoA) reductase.

24. A treatment device for treating a treatment site of a mammal, the mammal having a vessel that includes a placement site having a preplacement site inner diameter prior to treatment, the treatment device comprising:
a frame that moves from a contracted first configuration to an expanded second configuration, the frame delivering a treatment that includes at least one of ApoA1 (natural occurring or synthetic versions) or ApoA2 (natural occurring or synthetic versions).

25. The treatment device of claim 24 wherein the frame has a frame outer diameter at the second configuration that is within approximately twenty percent of the preplacement site inner diameter.

26. A method for treating a treatment site of a mammal, the mammal having a vessel including a placement site having a preplacement site inner diameter prior to treatment, the method comprising the steps of:
positioning a frame inside the vessel at the placement site; and
expanding the frame so that the frame has a frame outer diameter that is approximately equal to the preplacement site inner diameter.

27. The method of claim 26 wherein the step of expanding includes expanding the frame so that the frame outer diameter is within approximately one percent greater than the preplacement site inner diameter.

28. The method of claim 26 wherein the step of expanding includes expanding the frame so that the frame outer diameter is within approximately five percent greater than the preplacement site inner diameter.

29. The method of claim 26 wherein the step of expanding includes expanding the frame so that the frame outer diameter is within approximately ten percent greater than the preplacement site inner diameter.

30. The method of claim 26 wherein the step of expanding includes expanding the frame so that the frame outer diameter is within approximately twenty percent greater than the preplacement site inner diameter.

31. The method of claim 26 further comprising the step of delivering a treatment from the frame to the treatment site.
